(19) United States
(12) Patent Application Publication
(10) Pub. No.: US 2015/0329814 A1
(43) Pub. Date: Nov. 19, 2015

(54) Apparatus for long-term uninterrupted safe culturing of embryos, biological specimens in a controlled environment having improved imaging capabilities

(71) Applicant: Michael D. Cecchi, Madison, CT (US)

(72) Inventor: Michael D. Cecchi, Madison, CT (US)

(21) Appl. No.: 14/120,322

(22) Filed: May 16, 2014

(51) Int. Cl. C12M 1/32; C12M 1/34; C12M 1/00

(52) U.S. Cl. CPC C12M 23/12; C12M 23/50; C12M 41/00

(57) ABSTRACT

The invention relates to culturing dishes, which will be used for long-term, uninterrupted culturing of embryos in a controlled environment. The dishes will provide the embryos with ample amounts of culture media, with an oil overlay, to protect them from environmental impact, from contaminants, and will hold the embryos in a safe environment which will protect them from the effects of disruptions such as handling. The dishes contain uniquely designed specimen well bottoms which will allow accurate and repeated locating of the embryos as well as improving the imaging quality and the use of higher magnifications. This allows continual and repeated imaging of the embryos, data logging, aiding in the selection process, and will give the embryos a greater likelihood of improved growth, reimplantation capabilities and will increase successful outcomes, results and a greater likelihood of live births in this IVF environment.

### TECHNICAL FIELD

This invention relates to an apparatus and method for the long-term, uninterrupted, culturing of specimens, such as cells, stem cells, oocytes and embryos in an incubated environment with imaging and evaluation capabilities. The invention's apparatus results in an enhanced ability to allow better viewing and imaging of the specimens using a microscope or camera system, and increase the likelihood of rapid location of the embryos within a designated well; and allows the use of greater magnification when imaging or viewing. The invention, overall, gives the ability of long term, uninterrupted culturing of the specimens within this controlled environment, throughout all growth stages, and up to reimplantation in the case of embryos. The apparatus includes features, which will protect the specimens from environmental factors and conditions, such as volatile organic compounds, which may be within the environment, and outside influences such as disturbances, earthquakes or mishandling by the users. The specimens, such as embryos, will be protected from these conditions due to the awareness of possible problems, and including features, which will overcome these problems. The invention protects the specimens and allows continued growth with desirable outcome and results. This will give the users a greater likelihood of desired pregnancies and live births.

### BACKGROUND ART

Currently there are many petri dishes used in the culturing of oocytes, embryos and stem cells. The majority of petri dishes used in culturing have large open flat surfaces.

The most widely used method for culturing oocytes and embryos is to use a petri dish, which has flat surfaces, and may be about thirty mm dish or about sixty mm dish. The users will create micro droplets to culture the embryos in. These droplets are simply droplets of culture media on the floor of the dish, surrounded by a mineral oil. The mineral oil is applied to try to protect from embryo dehydration and temperature changes.

The problem facing this micro droplet system is the fact that the droplet may not be a sufficient amount of media for the proper growth of the embryo, the oil may permeate the droplet and contaminate the embryo, and the droplet may collapse. The collapse would not be beneficial to the embryo and may result in permanent damage to the embryo and may cause the embryo to arrest, thereby causing the embryo to not mature and not be implantable.

An additional problem currently faced by the use of current petri dishes is the disturbance to the embryo due to the mishandling of dish by the user, a disruption to the embryo caused by the shifting within the incubator or a major disruption caused by an earthquake or by other means.

An additional problem with current dishes is that the bottom is flat, may have wells and may not have wells, which will have a flat bottoms. The flat bottom allows the embryo to rest anywhere in the droplet or well, which my be against the dish wall or well wall and this will not allow the user to be able find the embryo quickly and easily and may distort the shape of the embryos. By resting against the wall and distorting the desired or roundish shape it may make visibility of certain features in the embryo impossible to view, or unable to view in its best positioning. One example would be the spindle of an embryo.

An additional problem with current dishes and imaging is that the embryo will rest anywhere in the micro drop or well and therefore will reduce the ability of the imaging device to use higher magnifications due to the necessity to support or view a larger area which the embryo is within.

Additionally, embryologists are concerned with the cost related to the pricing of media and oils and the inability to use less of these products which would result in savings to the users.

It would be highly desirable to provide an apparatus and system which would provide an embryo with the full desired amounts of media to support the growth and to maintain an adequate supply of oil above the embryo to protect it from environment conditions such as VOC's within the incubator, evaporation, and contamination.

It would be highly desirable to provide an apparatus and system which will help protect the embryo, allow it to be highly visible, with the use of a microscope or camera, and allow a variable range of magnifications.

It would be highly desirable to provide an apparatus and system which will allow the long-term culturing of embryos, provide improved images of their development; and to be able to implant the most desirable embryos.

It would be highly desirable to provide an apparatus and system which will allow a higher resolution in imaging and a more concise series of these images, which will allow better evaluation, better embryo selection, and a better likelihood of implanting more capable embryos for growth, pregnancy and a greater birth rates.

### DISCLOSURE OF THE INVENTION

This invention relates to an improved dish for growing specimens such as embryos, for an extended period of time, allowing excellent visibility for observation with a microscope or camera, allowing time lapse photography, providing better and increased magnification of the embryos so as to allow their monitoring; and increasing the likelihood of more pregnancies and ultimately more live births.

The device is a dish, or a dish-like configuration, which has several unique features, and embodiments, which will make up the dish and provide improved performance and results.

One example of an embodiment of the dish, is a culturing dish with an outer wall diameter of about fifty to sixty mm, and a smaller inner ring of about thirty to forty mm in diameter, along with smaller wells of about four to seven mm in diameter, within the inner ring to hold the embryos in culture media. The inner ring may be filled with mineral oil, which may also cover the smaller culturing wells. This inner ring which is within a larger outer walled dish, allowing greater safety in handling the dish while taking advantage of a smaller configuration walls to culture to help control the amount of media and oils used.

The dish includes this inner ring to contain the oil, and to allow this inner area to be a safe zone for the protection of the embryos. Within this area are small, micro sized wells of about seventy five to one hundred twenty five μl in volume, which will allow the use of desirable amounts of media to be determined and used, and allow the embryos to be safety held within. These wells are having slanted exterior walls to allow the embryos to stay away from the walls and to be easily found and have less likelihood of attaching themselves to the wails. The micro wells have sloping, bottom walls as opposed to a flat bottom, which reduce the shadows in the dish, when observed through a microscope.

The area between the inner wall and outer wall of the dish will allow the end user to hold additional media or oils for temperature stability of the dish or to temporarily hold additional specimens. It will also be a safety zone if any specimens or material are expelled from the inner ring. The inner ring allows the user to utilize the dish as if it was a thirty five mm petri dish, which is commonly used by embryologist, but have the stability of a fifty five mm dish when handling.

The dish includes multiple micro wells for the holding of the embryos. These micro wells will have slanted inside exterior walls and a sloping bottom wall, in a conical shape, with the lowest point being the center of the well. In these wells embryos will migrate to the bottom, true center of the wells. This feature has several benefits. It allows for a clearer visibility, as there is less plastic between the low point of the well and bottom of the dish. This is important as the embryologist use an inverted microscope a view embryos from the bottom of the dish. This feature also allows the embryo to be easily located as they will be at the same point in the dish for viewing.

This feature allows a microscope or camera to focus in on a much smaller initial point of focus, gain better imaging and allows a greater magnification due to the embryo being in a more concise location. Embryos are approximately one hundred forty ul in size. A conventional well or micro drop is much larger and may be ten times that diameter. Accordingly, other systems must use the larger dimension to begin with and then try to magnify from that size, which limits the size of the ultimate image and at what magnification, as the embryo may be anywhere within that field. This invention, by allowing the embryo to settle in the true center of the well allows the camera or microscope to start with about a one hundred forty to two hundred ul field, and to magnify from that, allowing much greater magnification, with the capable image able to be produced on a screen or picture. In a preferred embodiment of this invention the dish has twelve wells and therefore twelve concise locations for the microscope or camera to focus on, instead of possibly hundreds of locations with the conventional petri dishes.

The dish will include imaging markers, orientation lines, numbers and/or letters to allow the users to orient the dish and to be able to monitor and locate embryos easily and repeatedly find them when needed. The size and height of the wells allow easy access and hold the amount of media desired.

The dish allows the embryos to be held in concise amounts of media, overlaid by oil for protection, in precise focal locations. The dish includes an inner ring which allows less movement or sloshing to the media and oil if the dish is disrupted or moved. This multi layer of protection will help protect the embryos from such factors of the dish being mishandled by the user, a disruption on the dish due to jostling of the dish on a shelf from the bumping of an incubator, or major disruptions, such as topical storms or earthquakes.

A second embodiment of the invention contains a configuration of a dish with four larger wells and four smaller wells for the continual, uninterrupted culturing of embryos for the desired time, within a controlled environment.

An example of this embodiment is a dish with four large wells and four smaller wells. Each larger well is about one to one and one half centimeters in diameter, which will hold up to two hundred fifty to three hundred micro liters of culture media. These larger wells contain culture media, which will be covered or overlaid with a layer of mineral oil. In this embodiment the wells are approximately two to five millimeters in height.

The large volume of culture media, about two hundred fifty to about three hundred ul, and the additional amount of the mineral oil will contain the culture media within in the wells. The larger volume of the culture media will allow a longer culturing time of these embryos and the users may choose to make none or few changes in replenishing the culture media during the maturation of the embryos. In this example, the six wells will contain the slanted well walls and the conical bottom of the well, which will give the dish a more precise focal point and dearer images and the ability to user higher magnifications for better evaluation and choice for implantation. In this example the inclusion of six wells gives the microscope and camera six concise locations for finding and focusing in on embryos or other specimens.

In this embodiment the wells will hold larger mounts of culture media and will provide the oil overlay and safety features needed for the protection of the embryos for long term culturing. It will include the unique features of the invention for imaging and resolution of the embryos.

In both embodiments, the dishes have a concise number of focus points which will allow the camera or microscope to focus with less overall movement, move fewer times and therefore will allow the mechanism to incur less wear and will extend the life of the mechanism.

The invention and its embodiments will provide a safety factor and feature to the embryos, if in fact they need to be evacuated due to major disruptions such as power outages, brush fires, storms or earthquakes.

It is therefore an object of this invention to provide a method and apparatus of the character described, which will allow more concise imaging of the specimens as they are cultured through a growth period.

It is another object of this invention to provide a method and apparatus of the character described which will allow long term uninterrupted embryo culturing.

It is a further object of this invention to provide a method and apparatus of the character described which will give the embryos safety from outside influences, such as airborne contaminants, disruptive movement, earthquakes and the like.

It is yet another object of this invention to provide a method and apparatus of the character described which combine multiple growth and safety features within a single embodiment.

These and other objects and advantages of the invention will become more readily apparent from the following detailed description of the invention, when taken in conjunction with the accompanying drawings, in which:

### BRIEF DESCRIPTION OF THE DRAWINGS

FIG. 1 is a top plan view of a culture dish formed in accordance with this invention;

FIG. 2 is a schematic side view of a portion of the dish of FIG. 1;

FIG. 3 is a schematic side view the dish of of FIG. 1 illustrating the method of use and the invention's safety features; and

FIG. 4. Is a top plan view similar to FIG. 1, but showing another embodiment of the compartmentalized embryo growth and culturing dish formed in accordance with this invention.

### DETAILED DESCRIPTION OF THE INVENTION

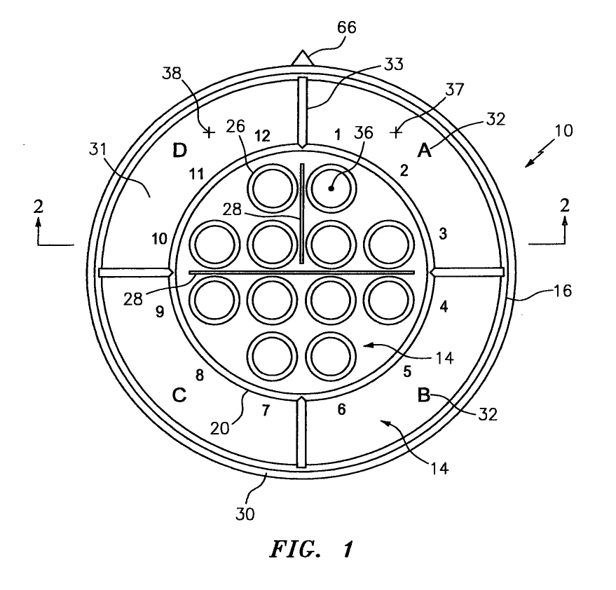

FIG. 1 is a top plan view of an oocyte, embryo and stem cell specimen growth dish, denoted generally by the numeral 10, which is formed in accordance with this invention. The dish 10 has an outer wall 16 and a bottom wall 14. The bottom wall 14 is flat, and has a small raised portion 30 below the outer wall 16, which is placed in the outer perimeter of the dish 10 and which will keep the bottom wall 14 slightly elevated above a surface it may be placed on, thereby preventing the bottom wall 14 from being scratched by the surface it is placed on. This will keep the viewable portion from being scratched and maintain a clear view. The user will most likely view the specimens from under the dish 10 with a microscope or camera. The dish 10 has an inner well wall 20, which may be used to contain oil in an outer well area 31 which is divided into four quadrants. This outer well area 31 may be used for holding extra oil, which may help to maintain the overall temperature of the dish 10, or it may store extra media, specimens or additional materials which create a safety zone in case embryos may be expelled from the inner well.

The dish 10 contains several orientation indicators 66, 37 and 38 which allow the user to know where he or she is within the dish 10 while using the microscope or camera, and additionally be able to record the positioning of the embryos in the dish 10 and to be able to maintain a system for monitoring the embryos with the ability to locate specific ones of the embryos each time the embryos are viewed. The orientation bar 66, allows a user to know this is the “top” of the dish 10, and will allow the dish 10 to be placed in a slot or holding area within an incubator, that will insure the dish 10 is correctly oriented in relationship to the microscope or camera positioning, and will assure the user the dish is in the same orientation at each imaging time, and will enable the microscope or camera to find and image of all the wells within the dish 10. Orientation indicia 37 and 38 are within the floor 14 of the dish 10. These indicia 37 and 38 may be encoded to identify the dish 10 and the donor and/or recipient of the embryos.

The dish has strips 28, which form a small ridge in the dish 10, these strips 28 are seen to go from left to right, and from the middle to the top of the dish. This is to establish a directional guide for the orientation of the dish 10.

The dish 10 contains numbers 34 which are, in this example, from one to twelve, similar to the configuration of clock face. These numbers 34 will give the user the orientation of the dish 10, like a clock face and allow the user to note which well 26 specific embryos are stored in. The dish has letters 32, signified as A, B, C and D, which will give the outer wells 31 designations, to allow for identification for these outer wells 31.

Included in the dish 10 are twelve smaller wells 26 which will be used to hold the embryos during culturing. The wells 26 are recessed in their center so that the well's floor 36 is conical, with the outer portion of the floor 36 being slighter raised with respect to the center. The conical shape causes the embryos to rest at the center, that being the lowest point in the wells 26. This recessed center of the floor 36, will allow the user to find the embryos more quickly and more easily, and to know that each time, the embryos will rest at this low point. This recessed center also allows better visibility, increased magnification capabilities, and more accurate imaging.

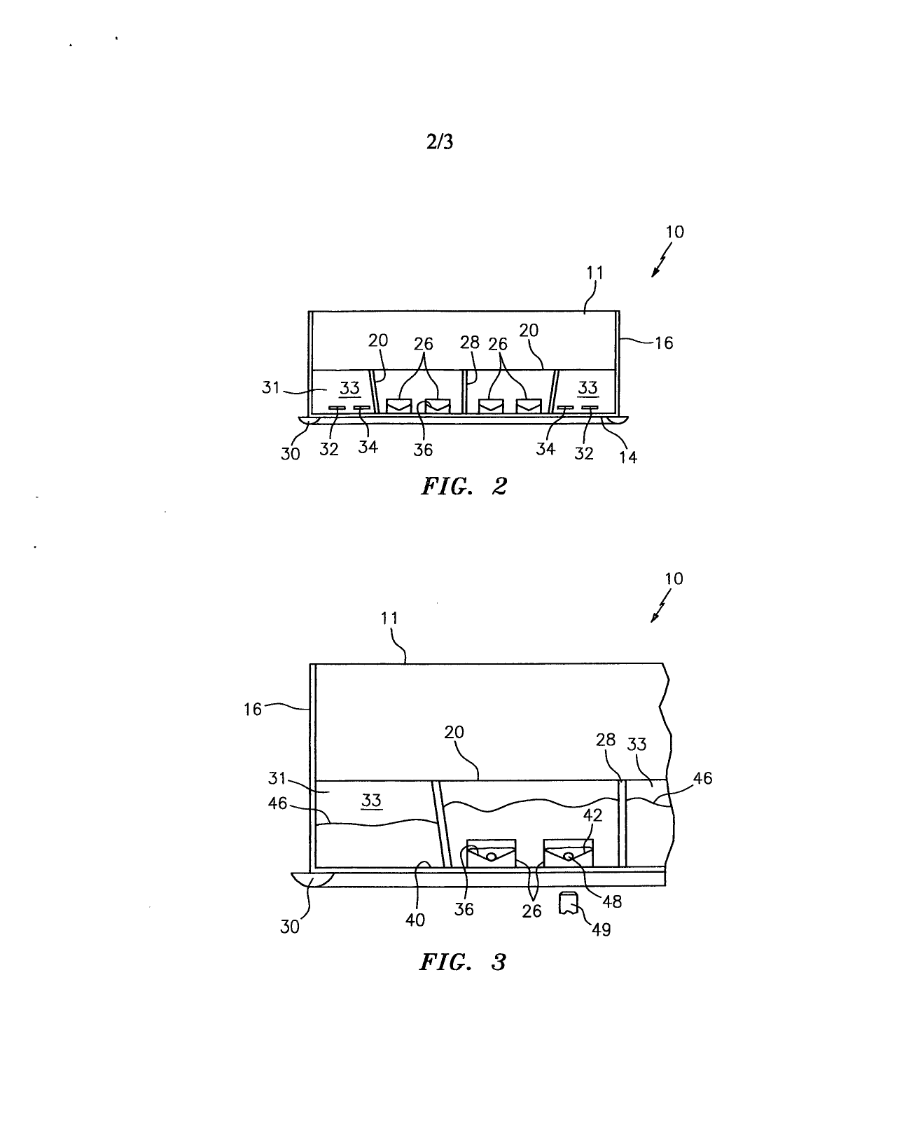

FIG. 2 shows a side sectional view of the dish 10. FIG. 2 shows the dish 10, the outer wall 16, the top edge 11 of outer wall 16, the bottom wall 14, and the additional wall 20, which create the outer wells 31. Also shown are the raised bottom edge 30 which keeps the bottom surface 40 slightly elevated to prevent scratching; the smaller specimen wells 26, the numbers 34 and letters 32. This figure illustrates the height relationship of the outer wall 16, inner walls 20 and micro wells 26. In the side view of the wells 26, the conical floor 36, which results in a recessed center where the embryo will reside, is dearly illustrated.

The dish 10 utilizes the inner wall 20, along with the small wells 26, with the conical bottoms 36 which cause the embryos to settle at the lowest points in the dish 10, to protect the embryo from damage, disturbance and outside influences. This provides a far safer environment for the embryos than if they were held in a micro drop.

FIG. 3 illustrates the structural significance of the dish 10 with its resultant method of use, to show the significant safety features of the dish 10. FIG. 3 shows a partial side view of the dish 10, illustrating the method of use of the dish 10 and the safety features created by it to protect the embryos. The methods of the invention illustrate the safety features and how they protect the embryos. The components of the dish 10 are shown: the dish 10; the top 11 of the dish 10; the outer wall 16; the inner wall 20; the walls between the inner ring and outer wall 22; the ring 30 on the bottom; the conical bottoms 36 to the wells; the embryos 48; the media solution 42 in the wells; and the mineral oil 46.

In this example, the over all dimensions of the dish are: the wells 26 are approximately one half to two millimeters in height; the inner wall 20 is approximately three to six millimeters in height; the outer wall 16 is approximately eight to twelve millimeters in height, the dimension of the outer well 31 is approximately fifty to sixty millimeters in diameter; the inner ring is approximately thirty to thirty five millimeters in diameter; and the smaller wells 26 are about three to six millimeters in diameter. As shown in FIG. 3 the embryos 48 are resting at the lowest point of the conical bottoms 34 within the wells 26. The wells 26 are filled with approximately twenty ultra liters of culture media. In comparison a micro droplet will be of approximately five ultra liters of media. This supplies the embryos with ample amounts of culture media for maturation. The inner ring 20 is filled close to its top with mineral oil 46. Mineral oil is also placed in a small quantity in the wells 31, formed by the inner walls 20 and outer walls 16. This mineral oil 46 helps to maintain a constant temperature in the dish, when inside the incubator, and when removed from the incubator for observation and manipulation. FIG. 3 includes a conceptual fragmented view of a specimen imaging instrument 49, such as a microscope or camera for recording images of the specimens 48 during the culturing period thereof.

This additional embodiment exemplifies the safety of the embryo in this unique invention. As the embryo 48 is resting at the base of the well 26, covered with culture media 42, the walls of the well 26 will keep the media from dispersing; the mineral oil 46 above and around the culture media 42 will hold the media in place, using fluid dynamics. Mineral oil 46 will sit on top of water thereby forcing the culture media to the bottom of the dish wells 26 and the mineral oil which surrounds the wells 26 prevents the culture media from spilling over the edges of the wells 26. The mineral oil 46 is approximately four times the height of the culture media 42. This significant difference keeps the culture media in place. The dimension of the inner wall at thirty two millimeters will keep the oil from moving about the dish creating any type of major disturbance to the culture media 42 thus keeping the embryos 48 safely resting at the bottom of the wells 26.

This method of use and the structural elements of the dish have been shown to keep the embryos safely in place during handling and disturbances, and will provide the user with security when handling the dish or in the case of major disturbances such as storms, blackouts, hurricanes, earthquakes and the like.

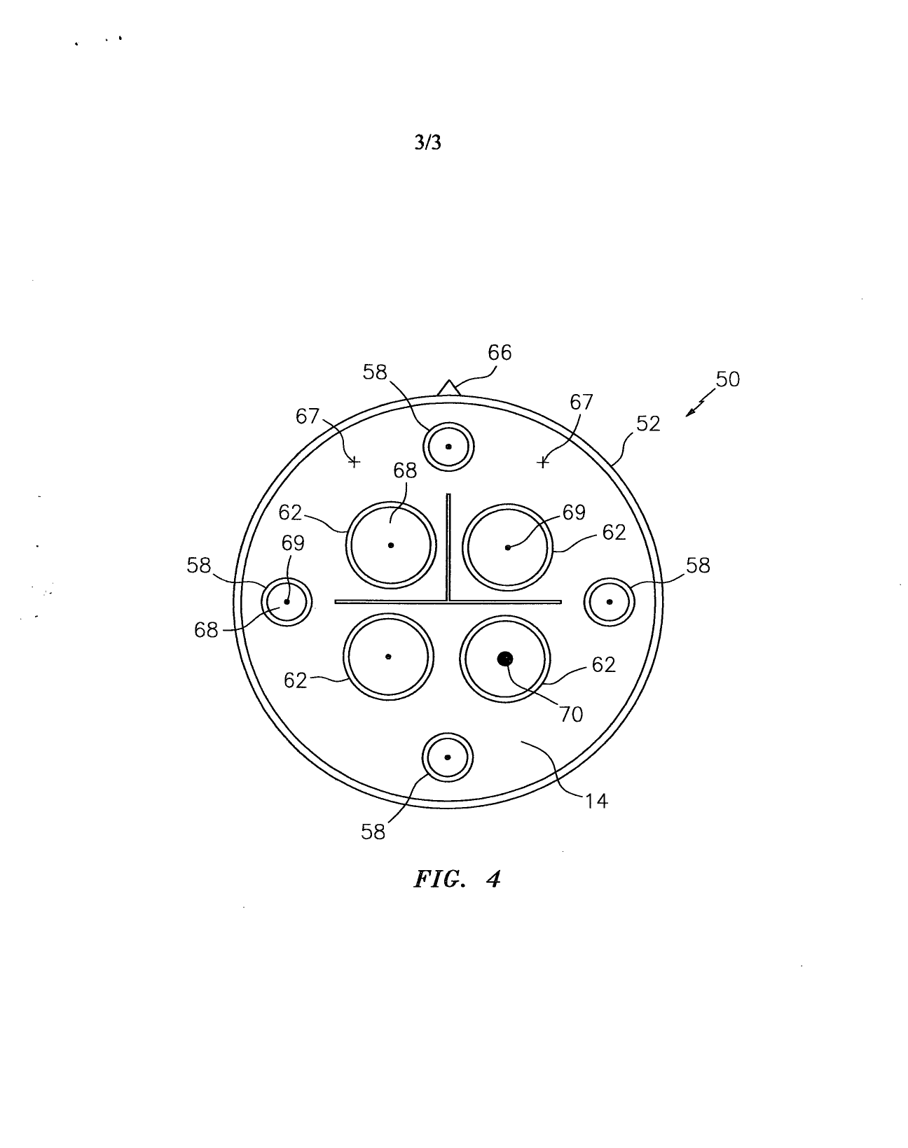

FIG. 4 Is a top plan view of another embodiment of the invention. This dish 50 is composed of an outer wall 52; four inner wells 62 and four outer wells 58 with conical bottoms 68. There is an orientation bar 66 at twelve o'clock in this example. This orientation bar 66 allows a user to consider this as the “top” of the dish 50, and will allow the dish 50 to be placed in a slot, or holding area of the dish 50 that will ensure that the dish is correctly oriented in relationship to the microscope or camera positioning, and will assure the user that the dish 50 is in the same orientation each time embryo readings are taken, and will assist the microscope or camera in finding and imaging all the wells within the dish. The method for utilizing this dish 50 is to provide larger wells which will contain larger amounts of culture media of up to three hundred micro liters, which will allow the culturing of the embryos for a longer period of time and will allow the user to perform alternate procedures, such the vitrification of oocytes and embryos, or to manipulate the embryos, or perform such procedures and ICSI, PCR or cellular manipulation or extractions.

The dish 50 will contain at least two indicators 67 in its bottom wall which will allow a user or automatic microscope system to find these indicators 67 and thereby orient itself relative to the rest of the dish 50.

The large volume of culture media, about two hundred to about three hundred ul, and the additional amount of mineral oil, will retain the culture media in the wells 62 during handling or disturbances. The larger volume of the culture media will allow a longer culturing time of these embryos and the users may choose to make fewer, or no, replenishing of the culture media during the maturation of the embryos.

All eight wells in this example will contain slanted well walls 68 and the unique conical bottoms 69 of the wells will give the dish a precise focal point and clearer embryo images, and the ability to use higher magnifications for better embryo evaluation and choices for reimplantation. The embryos 70 will rest at the lowest point for precise imaging.

The method of use and structural features of this dish 50 create a safer and longer maturation period between media changes and mineral oil. In this dish environment the embryos will be protected from external elements and disruptions of moving, handling, earthquakes, storms and the like.

Since many changes and variations of the disclosed embodiment of the invention may be made without departing from the inventive concept, it is not intended to limit the invention except as required by the appended claims.

What is claimed is:

1. A biological specimen culturing dish for culturing biological specimens such as oocytes, embryos, stem cells, tissue cells and the like, said culturing container including:
a) a bottom wall and an outermost side wall;
b) an inner wall which separates said dish into an inner area and an outer area, said outer area including said outermost side wall;
c) at least one well in said inner area containing a specimen; said well having a conical shaped bottom whereby said specimen will be held within the conical bottom's lowest point at the true center of said well;
d) said dish including a ring-shaped raised portion on the periphery of said dish bottom wall, said raised portion being operative to elevate the lower outside surface of said bottom wall above any support surface said dish is placed on so as to prevent said lower outside surface of said dish from being scratched by a support surface; and
e) an imaging instrument disposed below said support surface, said imaging instrument being operative to periodically view and record images of said specimen in said said well.

2. The container of claim 1 further including an orientation bar formed on the outer surface of said outermost side wall, said orientation bar providing means for physically orienting said dish relative to said imaging instrument.

3. The container of claim 2 further including additional orientation means on said dish, said additional orientation means being angularly offset from said orientation bar.
